(19) United States
(12) Patent Application Publication
(10) Pub. No.: US 2026/0216502 A1
(43) Pub. Date: Jul. 30, 2026

(54) SYSTEMS AND METHODS FOR EXTRACTING AN ELECTRODE LEAD

(71) Applicant: Mainstay Medical Limited, Dublin (IE)

(72) Inventors: Michael DAHL, Eden Prairie, MN (US); Jason SKUBITZ, Arden Hills, MN (US)

(73) Assignee: Mainstay Medical Limited, Dublin (IE)

(21) Appl. No.: 19/573,921

(22) Filed: Mar. 20, 2026

Related U.S. Application Data
(63) Continuation of application No. 18/186,149, filed on Mar. 18, 2023, now Pat. No. 12,582,816.
(60) Provisional application No. 63/269,756, filed on Mar. 22, 2022.

(51) Int. Cl. A61N 1/05; A61B 17/16; A61M 29/02

(52) U.S. Cl. CPC A61N 1/0558; A61B 17/1671; A61M 29/02; A61M 2210/1003

(57) ABSTRACT

Systems and methods are provided for extracting an implanted lead from a patient's body. The system may include a dilator and a trephine for extracting the electrode lead. A sheath may be included that is advanced over the proximal end of an electrode lead until the distal end of the sheath abuts a collar of the electrode lead coupled to proximal fixation elements. The dilator may be rotatably and axially advanced over the sheath until the distal end of the dilator collapses the proximal fixation elements within a lumen of the dilator. The trephine may then be rotatably and axially advanced over the dilator such that a cutting edge of the trephine cuts tissue surrounding distal fixation elements of the electrode lead to thereby dislodge the electrode lead.

### CROSS-REFERENCE TO RELATED APPLICATIONS

This application is a continuation of U.S. patent application Ser. No. 18/186,149, filed Mar. 18, 2023, now U.S. Pat. No. 12,582,816, which claims priority to U.S. Provisional Patent Application Ser. No. 63/269,756, filed Mar. 22, 2022, the entire contents of each of which are incorporated herein by reference.

### FIELD OF USE

The present disclosure is directed to a lead extraction system and methods for extracting an electrode lead implanted within a patient.

### BACKGROUND

The human back is a complicated structure including bones, muscles, ligaments, tendons, nerves and other structures. The spinal column has interleaved vertebral bodies and intervertebral discs, and permits motion in several planes including flexion-extension, lateral bending, axial rotation, longitudinal axial distraction-compression, anterior-posterior sagittal translation, and left-right horizontal translation. The spine provides connection points for a complex collection of muscles that are subject to both voluntary and involuntary control.

Back pain in the lower or lumbar region of the back is common. In many cases, the cause of back pain is unknown. It is believed that some cases of back pain are caused by abnormal mechanics of the spinal column. Degenerative changes, injury of the ligaments, acute trauma, or repetitive microtrauma may lead to back pain via inflammation, biochemical and nutritional changes, immunological factors, changes in the structure or material of the endplates or discs, and pathology of neural structures.

The spinal stabilization system may be conceptualized to include three subsystems: 1) the spinal column, which provides intrinsic mechanical stability; 2) the spinal muscles, which surround the spinal column and provide dynamic stability; and 3) the neuromotor control unit, which evaluates and determines requirements for stability via a coordinated muscle response. In patients with a functional stabilization system, these three subsystems work together to provide mechanical stability. It is applicant's realization that low back pain results from dysfunction of these subsystems.

The spinal column consists of vertebrae and ligaments, e.g. spinal ligaments, disc annulus, and facet capsules. There has been an abundance of in-vitro work in explanted cadaver spines and models evaluating the relative contribution of various spinal column structures to stability, and how compromise of a specific column structure will lead to changes in the range of motion of spinal motion segments.

The spinal column also has a transducer function, to generate signals describing spinal posture, motions, and loads via mechanoreceptors present in the ligaments, facet capsules, disc annulus, and other connective tissues. These mechanoreceptors provide information to the neuromuscular control unit, which generates muscle response patterns to activate and coordinate the spinal muscles to provide muscle mechanical stability. Ligament injury, fatigue, and viscoelastic creep may corrupt signal transduction. If spinal column structure is compromised, due to injury, degeneration, or viscoelastic creep, then muscular stability must be increased to compensate and maintain stability.

Muscles provide mechanical stability to the spinal column. This is apparent by viewing cross section images of the spine, as the total area of the cross sections of the muscles surrounding the spinal column is larger than the spinal column itself. Additionally, the muscles have much larger lever arms than those of the intervertebral disc and ligaments.

Under normal circumstances, the mechanoreceptors exchange signals with the neuromuscular control unit for interpretation and action. The neuromuscular control unit produces a muscle response pattern based upon several factors, including the need for spinal stability, postural control, balance, and stress reduction on various spinal components.

It is believed that in some patients with back pain, the spinal stabilization system is dysfunctional. With soft tissue injury, mechanoreceptors may produce corrupted signals about vertebral position, motion, or loads, leading to an inappropriate muscle response. In addition, muscles themselves may be injured, fatigued, atrophied, or lose their strength, thus aggravating dysfunction of the spinal stabilization system. Conversely, muscles can disrupt the spinal stabilization system by going into spasm, contracting when they should remain inactive, or contracting out of sequence with other muscles. As muscles participate in the feedback loop via mechanoreceptors in the form of muscle spindles and golgi tendon organs, muscle dysfunction may further compromise normal muscle activation patterns via the feedback loops.

Trunk muscles may be categorized into local and global muscles. The local muscle system includes deep muscles, and portions of some muscles that have their origin or insertion on the vertebrae. These local muscles control the stiffness and intervertebral relationship of the spinal segments. They provide an efficient mechanism to fine-tune the control of intervertebral motion. The lumbar multifidus, with its vertebra-to-vertebra attachments is an example of a muscle of the local system. Another example is the transverse abdominus, with its direct attachments to the lumbar vertebrae through the thoracolumbar fascia. The thoracolumbar fascia is a deep investing membrane which covers the deep muscles of the back of the trunk. The thoracolumbar fascia includes superficial fascia and deep fascia. The superficial fascia is traditionally regarded as a layer of areolar connective or adipose tissue immediately beneath the skin, whereas deep fascia is a tougher, dense connective tissue continuous with it. Deep fascia is commonly arranged as sheets and typically forms a stocking around the muscles and tendons beneath it. Superficial fascia fibers run in the transverse direction, whereas deep fascia fibers run in a cranial-caudal direction.

The multifidus is the largest and most medial of the lumbar back muscles. It has a repeating series of fascicles which stem from the laminae and spinous processes of the vertebrae, and exhibit a constant pattern of attachments caudally. These fascicles are arranged in five overlapping groups such that each of the five lumbar vertebrae gives rise to one of these groups. At each segmental level, a fascicle arises from the base and caudolateral edge of the spinous process, and several fascicles arise, by way of a common tendon, from the caudal tip of the spinous process. Although confluent with one another at their origin, the fascicles in each group diverge caudally to assume separate attachments to the mamillary processes, the iliac crest, and the sacrum. Some of the deep fibers of the fascicles that attach to the mamillary processes attach to the capsules of the facet joints next to the mamillary processes. The fascicles arriving from the spinous process of a given vertebra are innervated by the medial branch of the dorsal ramus that issues from below that vertebra. The dorsal ramus is part of spinal nerve roots formed by the union of dorsal root fibers distal to the dorsal root ganglion and ventral root fibers. The dorsal root ganglion is a collection of sensory neurons that relay sensory information from the body to the central nervous system.

The global muscle system encompasses the large, superficial muscles of the trunk that cross multiple motion segments, and do not have direct attachment to the vertebrae. These muscles are the torque generators for spinal motion, and control spinal orientation, balance the external loads applied to the trunk, and transfer load from the thorax to the pelvis. Global muscles include the oblique internus abdominus, the obliquus externus abdmonimus, the rectus abdominus, the lateral fibers of the quadratus lumborum, and portions of the erector spinae.

Normally, load transmission is painless. Over time, dysfunction of the spinal stabilization system is believed to lead to instability, resulting in overloading of structures when the spine moves beyond its neutral zone. The neutral zone is a range of intervertebral motion, measured from a neutral position, within which the spinal motion is produced with a minimal internal resistance. High loads can lead to inflammation, disc degeneration, facet joint degeneration, and muscle fatigue. Since the endplates and annulus have a rich nerve supply, it is believed that abnormally high loads may be a cause of pain. Load transmission to the facets also may change with degenerative disc disease, leading to facet arthritis and facet pain.

Functional electrical stimulation (FES) is the application of electrical stimulation to cause muscle contraction to re-animate limbs following damage to the nervous system such as with stroke or spine injury. FES has been the subject of much prior art and scientific publications. In FES, the goal generally is to bypass the damaged nervous system and provide electrical stimulation to nerves or muscles directly which simulates the action of the nervous system. One lofty goal of FES is to enable paralyzed people to walk again, and that requires the coordinated action of several muscles activating several joints. The challenges of FES relate to graduation of force generated by the stimulated muscles, and the control system for each muscle as well as the system as a whole to produce the desired action such as standing and walking.

With normal physiology, sensors in the muscle, ligaments, tendons and other anatomical structures provide information such as the force a muscle is exerting or the position of a joint, and that information may be used in the normal physiological control system for limb position and muscle force. This sense is referred to as proprioception. In patients with spinal cord injury, the sensory nervous system is usually damaged as well as the motor system, and thus the afflicted person loses proprioception of what the muscle and limbs are doing. Functional electrical stimulation (FES) systems often seek to reproduce or simulate the damaged proprioceptive system with other sensors attached to a joint or muscle. FES has also been used to treat spasticity, characterized by continuous increased muscle tone, involuntary muscle contractions, and altered spinal reflexes which leads to muscle tightness, awkward movements, and is often accompanied by muscle weakness. Spasticity results from many causes including cerebral palsy, spinal cord injury, trauma, and neurodegenerative diseases.

Neuromuscular Electrical Stimulation (NMES) is a subset of the general field of electrical stimulation for muscle contraction, as it is generally applied to nerves and muscles which are anatomically intact, but malfunctioning in a different way. NMES may be delivered via an external system or, in some applications, via an implanted system. NMES via externally applied skin electrodes has been used to rehabilitate skeletal muscles after injury or surgery in the associated joint. This approach is commonly used to aid in the rehabilitation of the quadriceps muscle of the leg after knee surgery. Electrical stimulation is known to not only improve the strength and endurance of the muscle, but also to restore malfunctioning motor control to a muscle. See, e.g., Gondin et al., “Electromyostimulation Training Effects on Neural Drive and Muscle Architecture”, Medicine & Science in Sports & Exercise 37, No. 8, pp. 1291-99 (August 2005).

The goals and challenges of rehabilitation of anatomically intact (i.e., non-pathological) neuromuscular systems are fundamentally different from the goals and challenges of FES for treating spinal injury patients or people suffering from spasticity. In muscle rehabilitation, the primary goal is to restore normal functioning of the anatomically intact neuromuscular system, whereas in spinal injury and spasticity, the primary goal is to simulate normal activity of a pathologically damaged neuromuscular system.

U.S. Pat. Nos. 8,428,728 and 8,606,358 to Sachs, both assigned to the assignee of the present disclosure, and both incorporated herein in their entireties by reference, describe implanted electrical stimulation devices that are designed to restore neural drive and rehabilitate the multifidus muscle to improve stability of the spine. Rather than masking pain signals while the patient's spinal stability potentially undergoes further deterioration, the stimulator systems described in those applications are designed to reactivate the motor control system and/or strengthen the muscles that stabilize the spinal column, which in turn is expected to reduce persistent or recurrent pain.

While the stimulator systems described in the Sachs patents seek to rehabilitate the multifidus and restore neural drive, use of those systems necessitates the implantation of one or more electrode leads in the vicinity of a predetermined anatomical site, such as the medial branch of the dorsal ramus of the spinal nerve to elicit contraction of the lumbar multifidus muscle.

As discussed above, the deep back muscles are covered by the thoracolumbar fascia which comprises superficial fascia running in the transverse direction and deep fascia running in a cranial-caudal direction. There is a risk that electrode lead conductors may experience a tight bend near the location where the lead enters the thoracolumbar fascia when the lead is inserted within the body near the lateral edge of the spine. Such a tight bend may lead to dislodgement of the electrode lead and/or fracture, thereby preventing proper therapy delivery. The difference in directions of the superficial and deep fascia near the insertion site at the lateral edge of the spine may increase the risk of a high stress location on the lead, as described in U.S. Pat. No. 10,327,810 to Shiroff, assigned to the assignee of the present disclosure, and incorporated herein in its entirety by reference. Moreover, the muscles of the lower back are highly mobile and create an environment that can impose large mechanical stresses on electrode leads, resulting in a high risk of lead dislodgement and/or lead fracture. As a result, fracture-inducing shear forces as well as axial forces imposed on a conventional lead by the relative movement of the muscles overlying the back muscles targeted for stimulation may cause the lead to dislodge and/or fracture.

U.S. Pat. No. 9,999,763 to Shiroff, U.S. Pat. No. 9,950,159 to Beck, and U.S. Pat. No. 11,103,706 to Sachs, all assigned to the assignee of the present disclosure, and all incorporated herein in their entireties by reference, describe electrode leads having opposite-angled fixation elements, e.g., tines, for sandwiching an anchor site therebetween to anchor the electrodes of the electrode lead in or adjacent to tissue associated with one or more spine stabilizing muscles associated with local segmental control of a lumbar spine within a back of a patient. In addition, as the electrode leads may be implanted within the patient for a long period of time, tissue ingrowth may grow along the fixation elements, thereby adding further anchoring support to the electrode lead. However, there may be circumstances in which the electrode lead may need to be removed from the patient's body and/or replaced with another electrode lead. As the fixation elements may be embedded in tissue, simply retracting the electrode lead by applying a pulling force may fracture the lead elements, requiring a more invasive and time-consuming procedure for removal thereof.

Accordingly, there exists a need for a lead extraction tool that may safely remove an implanted electrode having fixation elements for anchoring the electrode lead at an anchor site.

### SUMMARY

The present disclosure overcomes the drawbacks of previously-known systems and methods by providing a lead extraction system for extracting an implanted electrode lead comprising a fixation element, e.g., proximal and distal fixation elements. The system may include a dilator having a lumen sized and shaped to receive a proximal end of the electrode lead therethrough such that the dilator is slideable along the electrode lead, wherein a distal portion of the dilator may be advanced distally to separate tissue surrounding the electrode lead. Moreover, the dilator may be advanced distally to collapse proximal fixation elements of the fixation element within the lumen of the dilator. The system further may include a trephine having a lumen sized and shaped to receive a proximal end of the dilator therethrough, wherein a distal portion of the trephine has a cutting edge that may be advanced distally to cut tissue surrounding the fixation element to thereby dislodge the implanted electrode lead. For example, the cutting edge of the trephine may be advanced distally to cut tissue surrounding distal fixation elements of the fixation element to thereby dislodge the electrode lead.

The system further may include a sheath having a lumen sized and shaped to receive a proximal end of the electrode lead therethrough such that the sheath is slideable along the electrode lead. A distal portion of the sheath may be advanced distally until it abuts a collar on the electrode lead that is coupled to the proximal fixation elements, thereby preventing further distal advancement of the sheath relative to the electrode lead. Accordingly, the lumen of the dilator may be sized and shaped to receive a proximal end of the sheath therethrough such that the dilator is slideable along the sheath. The sheath may have an outer diameter substantially equal to an outer diameter of the collar. The dilator may be rotated as the distal portion of the dilator is advanced distally to collapse the proximal fixation elements within the lumen of the dilator. Moreover, the distal portion of the dilator may be tapered. For example, a cross-sectional area of the distal portion of the dilator may decrease distally toward a distal end of the dilator.

The cutting edge of the distal portion of the trephine may include a plurality of proximally extending concave edges circumferentially disposed along a distal end of the trephine. In addition, the cutting edge of the distal portion of the trephine may be angled to facilitate cutting of the tissue surrounding the fixation element. Moreover, the trephine may be rotated as the cutting edge of the trephine is advanced distally to cut tissue surrounding the fixation element. The cutting edge of the distal portion of the trephine further may cut at least a portion of the fixation element. A proximal portion of the trephine may include a handle for facilitating rotation of the trephine by a user. At least one of the dilator or trephine may be formed of a rigid material, e.g., stainless steel. Moreover, a proximal portion of the dilator may include one or more textured surfaces configured to facilitate gripping of the dilator. In addition, the dilator may include an alignment marker disposed between two of the one or more textured surfaces, such that the alignment marker may facilitate alignment of the cutting edge of trephine and the distal portion of the dilator. For example, the alignment marker may include a groove extending circumferentially along an outer surface of the dilator. Alternatively, the alignment marker may include a smooth section between the two of the one or more textured surfaces. The system further may include an electrode lead having a fixation element, e.g., proximal and distal fixation elements, for anchoring the electrode lead in or adjacent to tissue associated with one or more spine stabilizing muscles associated with local segmental control of a lumbar spine within a back of a patient.

In accordance with another aspect of the present disclosure, a method for extracting an implanted electrode lead having a fixation element, e.g., proximal and distal fixation elements, from a patient's body is provided. The method may include advancing a dilator distally over a proximal end of the implanted electrode lead to thereby separate tissue surrounding the electrode lead. Moreover, advancing the dilator distally may include advancing the dilator distally over proximal fixation elements of the fixation element to thereby collapse the proximal fixation elements within the lumen of the dilator. The method further may include advancing a trephine distally over a proximal end of the dilator, such that a cutting edge at a distal portion of the trephine cuts tissue surrounding the fixation element, e.g., distal fixation elements of the fixation element, to thereby dislodge the implanted electrode lead; and removing the electrode lead from the patient's body. Prior to advancing the dilator distally over the proximal end of the implanted electrode lead, the method may include advancing a sheath distally over a proximal end of the implanted electrode lead until a distal end of the sheath abuts a collar of the proximal fixation elements. Accordingly, advancing the dilator distally over the proximal end of the implanted electrode lead includes advancing the dilator distally over the proximal end of the sheath.

Moreover, advancing the dilator distally over the proximal end of the implanted electrode lead may include rotating the dilator as the dilator is advanced distally. Additionally, advancing the trephine distally may include rotating the trephine as the trephine is advanced distally to cut the tissue surrounding the fixation element. The method further may include cutting at least a portion of the fixation element, e.g., the distal fixation elements, by advancing the trephine distally over the fixation element to facilitate dislodgement of the implanted electrode lead. In addition, the method may include removing the electrode lead and dilator proximally through the lumen of the trephine, and removing the trephine from the patient's body.

### BRIEF DESCRIPTION OF THE DRAWINGS

FIG. 1 is a schematic view of an exemplary embodiment of a lead extraction system constructed in accordance with the principles of the present disclosure.

FIG. 2 is a schematic view of an exemplary embodiment of an electrode lead constructed in accordance with the principles of the present disclosure.

FIG. 3A illustrates an exemplary sheath of the lead extraction system of FIG. 1, constructed in accordance with the principles of the present disclosure.

FIG. 3B is a close-up view of a distal portion of the sheath of FIG. 3A.

FIG. 4A illustrates an exemplary dilator of the lead extraction system of FIG. 1, constructed in accordance with the principles of the present disclosure.

FIG. 4B is a close-up view of a distal portion of the dilator of FIG. 4A, and FIG. 4C is a close-up view of an alignment marker at a proximal portion of the dilator of FIG. 4A.

FIG. 4D illustrates an alternative exemplary dilator constructed in accordance with the principles of the present disclosure.

FIG. 4E is a close-up view of an alignment marker at a proximal portion of the dilator of FIG. 4D.

FIG. 5A illustrates an exemplary trephine of the lead extraction system of FIG. 1, constructed in accordance with the principles of the present disclosure, and FIG. 5B is a cross-sectional view thereof.

FIG. 5C is a close-up view of a distal portion of the trephine of FIG. 5A.

FIG. 5D illustrates a handle at a proximal portion of the trephine of FIG. 5A.

FIG. 6 is a flow chart of exemplary method steps for extracting an implanted electrode lead in accordance with the principles of the present disclosure.

FIGS. 7A to 7I illustrate exemplary method steps for extracting an implanted electrode lead in accordance with the principles of the present disclosure.

### DETAILED DESCRIPTION

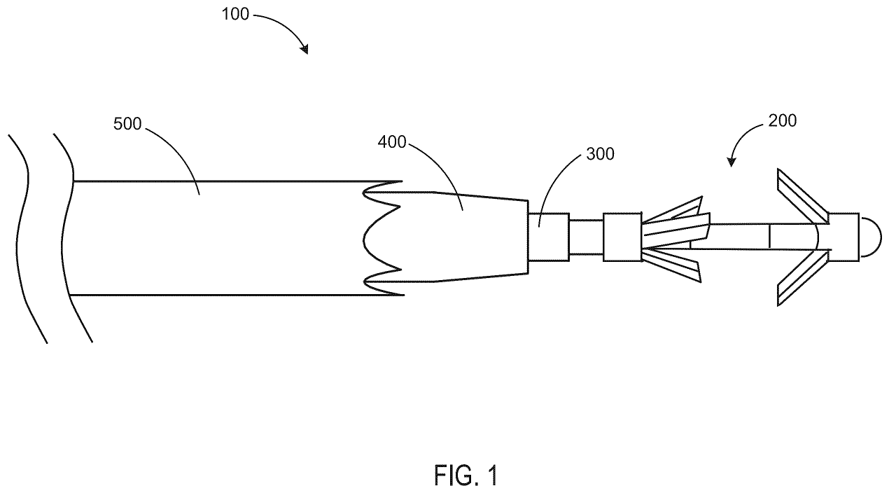

Referring now to FIG. 1, an exemplary lead extraction system is provided. In FIG. 1, components of the system are not depicted to scale on either a relative or absolute basis. As shown in FIG. 1, lead extraction system 100 may include sheath 300, dilator 400, and trephine 500 for extracting implanted electrode lead 200. As described in further detail below, electrode lead 200 may be implanted within a patient's body for providing electrostimulation therapy to treat, e.g., low back pain, by rehabilitating the spine stabilizing muscles associated with local segmental control of a lumbar spine within a back of the patient. As electrode lead 200 remains implanted within the patient, tissue ingrowth may develop along the fixation elements of electrode lead 200, thereby providing further anchoring support to electrode lead 200.

Under circumstances that may necessitate removal of implanted electrode lead 200 from the patient's body, lead extraction system 100 may be used to safely dislodge electrode lead 200 from the surrounding tissue for safe extraction of electrode lead 200 from the patient. For example, upon detachment of the proximal end of electrode lead 200 from a pulse generator, e.g., an implantable pulse generator or an external pulse generator, sheath 300 may be advanced over the proximal end of electrode lead 200 until a distal end of sheath 300 abuts a collar of electrode lead 200 coupled to the proximal fixation elements of electrode lead 200. Dilator 400 then may be advanced over the proximal end of sheath 300 and the proximal fixation elements while simultaneously rotated to thereby collapse the proximal fixation elements toward the lead body of electrode lead 200 within a lumen of dilator 400. Alternatively, sheath 300 may not be required, such that dilator 400 may be advanced directly over the proximal end of electrode lead 200 to collapse the proximal fixation elements of electrode lead 200. Trephine 500 may then be advanced over the proximal end of dilator 400 while simultaneously rotated to thereby cut tissue surrounding the fixation elements of electrode lead 200 and dislodge the distal fixation elements of electrode lead 200. Electrode lead 200, sheath 300, and dilator 400 may then be removed from the patient through the lumen of trephine 500, and trephine 500 may then be removed from the patient to complete the lead extraction process.

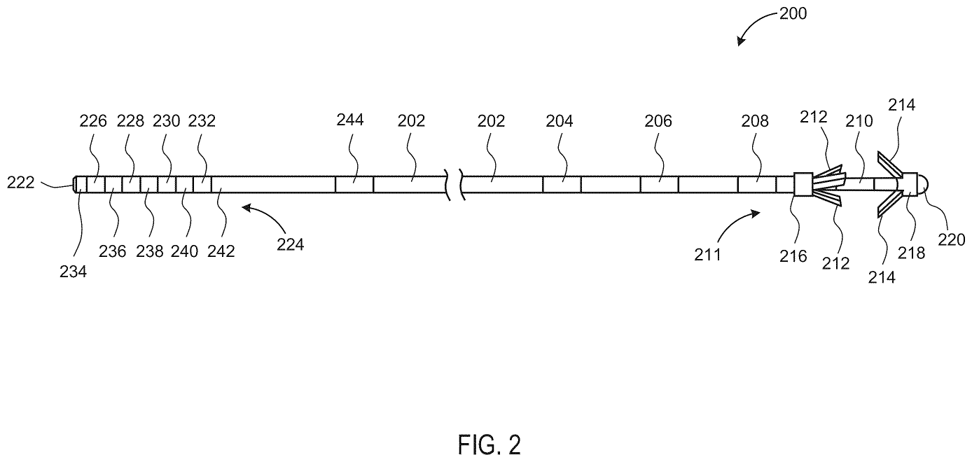

Referring now to FIG. 2, an exemplary electrode lead is provided. Electrode lead 200, e.g., an electrostimulation lead, may be coupled to a pulse generator, which may be actuated via an actuator, an external programmer, and a software-based programming system, as described in U.S. Pat. No. 9,950,159 to Beck, the entire contents of which are incorporated herein by reference. As shown in FIG. 2, electrode lead 200 includes lead body 202 having a plurality of electrodes, illustratively, electrodes 204, 206, 208, 210, disposed at distal region 211, the plurality of electrodes electrically coupled to a plurality of contacts, illustratively contacts 226, 228, 230, 232, disposed at proximal region 224, via a plurality of conductors. Electrode lead 200 is configured for implantation in or adjacent to tissue, e.g., nervous tissue, muscle, a ligament, and/or a joint capsule including tissue associated with local segmental control of the lumbar spine.

Lead body 202 is a suitable length for positioning the electrodes in or adjacent to target tissue while the pulse generator is implanted in a suitable location, e.g., the lower back, or alternatively, when the pulse generator is externally worn by the patient. For example, lead body 202 may be between about 30 and 80 cm in length, and preferably about 45 or about 65 cm in length. Lead body 202 is also of a suitable diameter for placement, for example, between about 1 and 2 mm in diameter and preferably about 1.3 mm. Electrodes 204, 206, 208, 210 may be configured to stimulate the tissue at a stimulation frequency and at a level and duration sufficient to cause muscle to contract and may be ring electrodes, partial electrodes, segmented electrodes, nerve cuff electrodes placed around the nerve innervating the target muscle, or the like. Electrodes 204, 206, 208, 210 are a suitable length(s) and spaced apart a suitable distance along lead body 202. For example, electrodes 204, 206, 208, 210 may be about 2-5 mm in length, and preferably about 3 mm, and may be spaced apart about 2-6 mm, and preferably about 4 mm. As will also be understood by one of skill in the art, an electrode lead may contain more or fewer than four electrodes.

As shown in FIG. 2, first and second fixation elements, e.g., proximal fixation elements 212 and distal fixation elements 214 may be coupled to lead body 202 at distal region 211 via first and second fixation rings/collars 216 and 218, respectively. First and second fixation elements 212 and 214 are configured to sandwich an anchor site, e.g., muscle, therebetween to secure electrode lead 200 at a target site without damaging the anchor site. In the illustrated embodiment, proximal fixation elements 212 are positioned between electrode 208 and distal most electrode 210 and distal fixation element 214 is positioned between distal most electrode 210 and end cap 220. The length of and spacing between the fixation elements is defined by the structure around which they are to be placed. In one embodiment, the length of each fixation element is between about 1.5-4 mm and preferably about 2.5 mm and the spacing is between about 2 mm and 10 mm and preferably about 6 mm. Proximal and distal fixation elements 212 and 214 are configured to collapse inward toward lead body 202 in a delivery state and to expand, e.g., due to retraction of a sheath, in a deployed state.

As shown in FIG. 2, proximal fixation elements 212 may be radially offset with respect to distal fixation elements 214. For example, proximal fixation elements 212 may be configured to be radially offset relative to distal fixation elements 214 by prefabricating at least one of first fixation collar 216 and second fixation collar 218 relative to lead body 202 such that at least one of proximal and distal fixation elements 212 and 214 is radially offset with respect to each other. For example, the projections of proximal fixation elements 212 may be radially offset relative to the projections of distal fixation elements 214 by, e.g., approximately 60 degrees. Moreover, proximal and distal fixation elements 212 and 214 may be formed of a flexible material, e.g., a polymer, and may be collapsible and self-expandable when deployed. For example, proximal and distal fixation elements 212 and 214 may collapse inward toward lead body 202 in a delivery state such that they are generally parallel to the longitudinal axis of lead body 202 within a sheath. In the delivery state, the radially offset proximal and distal fixation elements 212 and 214 need not overlap within a sheath.

Further, proximal and distal fixation elements 212 and 214 may expand, e.g., due to retraction of the sheath, in a deployed state. In the deployed state, proximal fixation elements 212 may be angled distally relative to lead body 202, and resist motion in the first direction, e.g., distally, and prevent, in the case illustrated, insertion of the lead too far, as well as migration distally. Distal fixation elements 214 may be angled proximally relative to lead body 202 and penetrate through a tissue plane and deploy on the distal side of the tissue immediately adjacent to the target of stimulation. Distal fixation elements 214 are configured to resist motion in the opposite direction relative to proximal fixation elements 212. This combination prevents migration both proximally and distally, and also in rotation.

While FIG. 2 illustrates proximal and distal fixation elements 212 and 214 on lead body 202, it should be understood that other fixation elements may be used to anchor electrode lead 200 at a suitable location including the fixation elements described in U.S. Pat. No. 9,079,019 to Crosby and U.S. Pat. No. 9,999,763 to Shiroff, both assigned to the assignee of the present disclosure, the entire contents of each of which are incorporated herein by reference.

Lead body 202 further may include stylet lumen 222 extending therethrough. Stylet lumen 222 may be shaped and sized to permit a stylet to be inserted therein, for example, during delivery of electrode lead 200. In one embodiment, end cap 220 is used to prevent the stylet from extending distally out of stylet lumen 222 beyond end cap 220.

As shown in FIG. 2, electrode lead 200 may include contacts 226, 228, 230, 232 at proximal region 224 separated along lead body 202 by a plurality of spacers 234, 236, 238, 240, 242, e.g., insulated tubing. Contacts 226, 228, 230, 232 may comprise an isodiametric terminal and are electrically coupled to electrodes 204, 206, 208, 210, respectively, via, for example, individually coated wires extending between the contacts and electrodes as described in further detail below. A portion of proximal region 224 may be configured to be inserted in the pulse generator. For example, a portion of proximal end 224 may be configured to be inserted in the pulse generator and set-screw retainer 244 may be configured to receive a screw from the pulse generator to secure the portion of electrode lead 200 within the pulse generator.

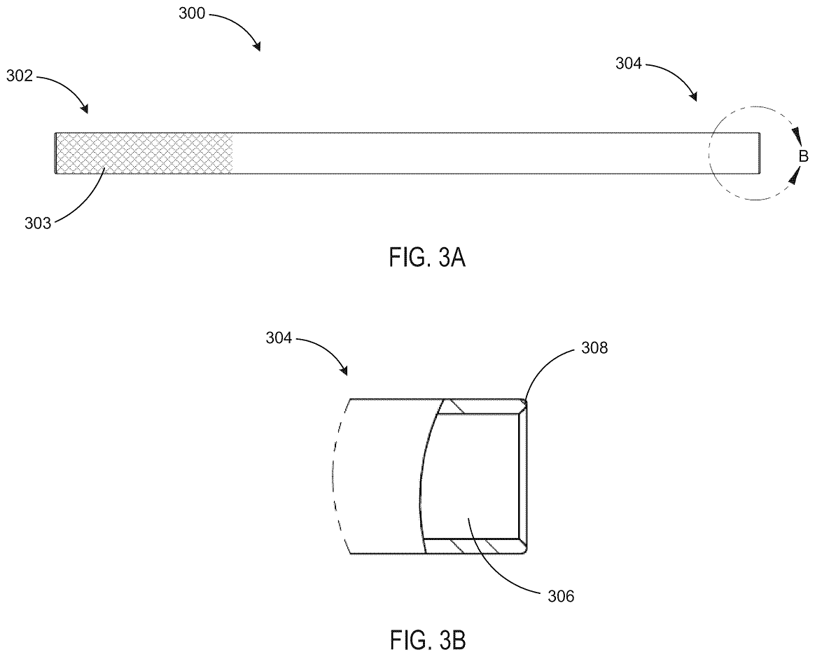

Referring now to FIGS. 3A and 3B, an exemplary sheath is provided. As shown in FIG. 3A, sheath 300 includes proximal portion 302 and distal portion 304. As shown in FIG. 3A, at least a portion of proximal region 302 may include textured surface 303 to facilitate gripping by the surgeon during the extraction process, e.g., to advance sheath 300 distally relative to electrode lead 200. For example, textured surface 303 may be debossed into the outer surface of proximal region 302 of sheath 300, e.g., via grit blasting or other machine methods known in the art. Moreover, textured surface 303 need not extend along the entire length of sheath 300. For example, textured surface 303 may extend about 2.5 inches along proximal region 302 of sheath 300. As will be understood by a person having ordinary skill in the art, textured surface 303 may extend along the outer surface of sheath 300 in any manner that permits the surgeon to effectively grip sheath 300 during the lead extraction procedure.

FIG. 3B is a close-up, partial cross-sectional view of distal region 304 of sheath 300, as indicated by circle B. Sheath 300 may be made of a rigid material, e.g., stainless steel, and have lumen 306 extending through an entire length of sheath 300. Lumen 306 is sized and shaped to receive the proximal end of electrode lead 200, such that sheath 300 is slidable over lead body 202 of electrode lead 200. For example, the proximal end of electrode lead 200 may be fed through lumen 306 at distal end 308 of sheath 300, such that sheath 300 may be advanced distally relative to electrode lead 200 until distal end 308 abuts collar 216 coupled to proximal fixation elements 212 of electrode lead 200. As shown in FIG. 1, the outer surface of sheath 300 may have a diameter that is substantially equal to collar 216 of electrode lead 200.

Referring again to FIG. 3B, distal end 308 of sheath 300 may not have sharp edges, such that distal end 308 may pass over lead body 202 of electrode lead 200 and through tissue while preventing damage to lead body 202 as sheath 300 is advanced over lead body 202. For example, distal end 308 may have a blunt and/or rounded distal edge. In some embodiments, sheath 300 may have a length such that when distal end 308 abuts collar 216, the proximal end of electrode lead 200 is exposed beyond proximal region 302 of sheath 300. Accordingly, when lead body 202 is received through sheath 300, sheath 300 provides stability to electrode lead 200 during the lead extraction process, e.g., via dilator 400 and trephine 500.

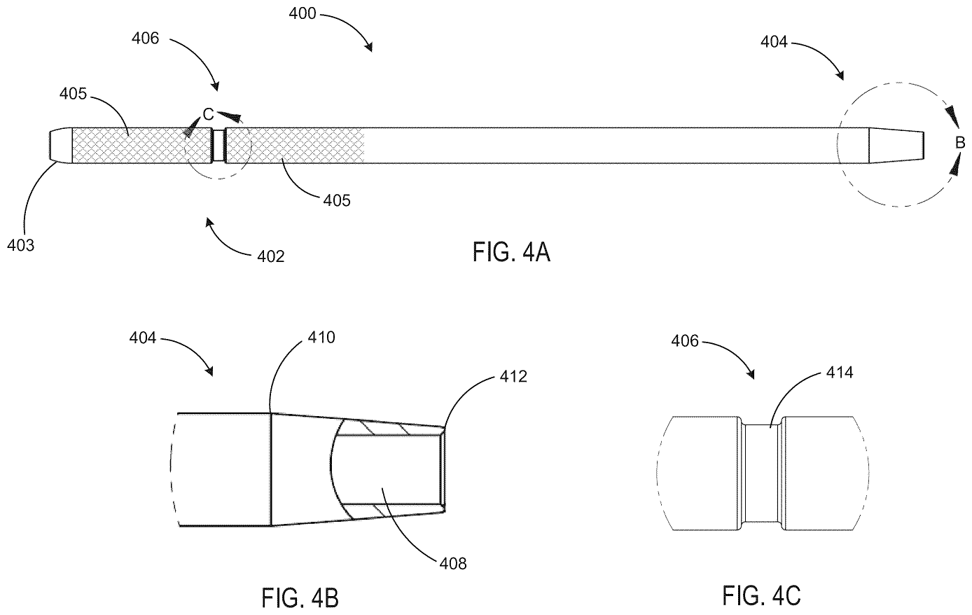

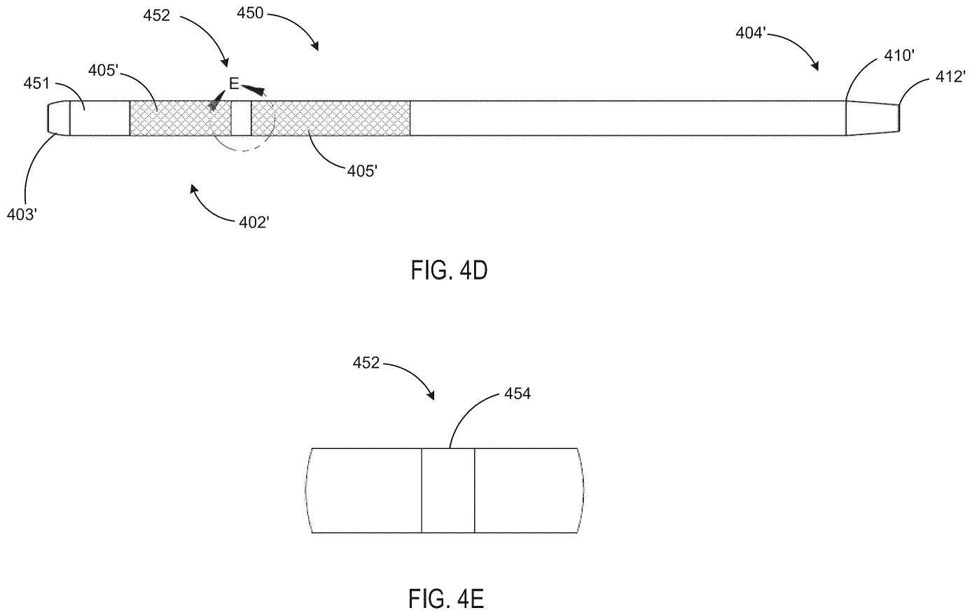

Referring now to FIGS. 4A to 4C, an exemplary dilator is provided. As shown in FIG. 4A, dilator 400 includes proximal region 402 and distal region 404. The proximal end of dilator 400 at proximal region 402 may include fillet 403, which may have a curved/angled outer surface to facilitate loading of trephine 500 over the proximal end of dilator 400, as described in further detail below. For example, the cross-sectional area of the outer surface of proximal region 402 of dilator 400 may decrease in the proximal direction toward the proximal end of dilator 400 at fillet 403. In addition, dilator 400 may include alignment marker 406 to visually indicate to an operator, e.g., a surgeon, alignment of the cutting edge of trephine 500 and distal region 404 of dilator 400. As shown in FIG. 4A, at least a portion of proximal region 402 may include textured surface 405 to facilitate gripping by the surgeon during the extraction process, e.g., to advance dilator 400 distally relative to electrode lead 200 while simultaneously rotating dilator 400. For example, textured surface 405 may be debossed into the outer surface of proximal region 402 of dilator 400, e.g., via grit blasting or other machine methods known in the art. Moreover, textured surface 405 need not extend along the entire length of dilator 400. For example, textured surface 405 may extend from about an inch from the proximal end of dilator 400 to about five inches from the distal end of dilator 400. As will be understood by a person having ordinary skill in the art, textured surface 405 may extend along the outer surface of dilator 400 in any manner that permits the surgeon to effectively grip dilator 400 during the lead extraction procedure.

FIG. 4B is a close-up, partial cross-sectional view of distal region 404 of dilator 400, as indicated by circle B. Dilator 400 may be made of a rigid material, e.g., stainless steel, and have lumen 408 extending through an entire length of dilator 400. Lumen 408 is sized and shaped to receive the proximal end of sheath 300, such that dilator 400 is rotatably slidable over sheath 300. For example, the proximal end of sheath 300 may be fed through lumen 408 at distal end 412 of dilator 400, such that dilator 400 may be advanced distally relative to sheath 300 and electrode lead 200. In addition, lumen 408 is further sized and shaped to receive collar 216 and proximal fixation elements 212 in a collapsed state therethrough. Accordingly, as distal region 404 of dilator 400 is advanced distally over collar 216 and proximal fixation elements 212, distal end 412 pushes against proximal fixation elements 212 to cause proximal fixation elements 212 to transition from the expanded, deployed state to the collapsed state against lead body 202 within lumen 408 of dilator 400, thereby dislodging proximal fixation elements 212 from the surrounding tissue. In some embodiments, sheath 300 may not be used during the lead extraction process, and accordingly, lumen 408 may be sized and shaped to receive the proximal end of electrode lead 200 directly, as well as collar 216 and proximal fixation elements 212.

As shown in FIG. 4B, distal region 404 of dilator 400 may be include a chamfered taper to facilitate dilation and passage of dilator 400 through tissue. For example, the cross-sectional area of distal region 404 may decrease from point 410 towards distal end 412 of dilator 400, such that the chamfered region of distal region 404 is tapered at a predefined angle. Notably, the predefined angle of the chamfered region of distal region 404 is not too large so as to not damage electrode lead 200 as dilator 400 is advanced over electrode lead 200, and to maintain enough line contact with the inner surface of trephine 500 so that the cutting edge of trephine 500 only interacts with tissue at the very distal end of dilator 400, as described in further detail below. The chamfered taper of distal region 404 is readably and noticeably distinguishable from curved fillet 403 of proximal region 402 by a surgeon, such that the surgeon will immediately know which orientation dilator 400 must be in to feed the proximal end of sheath 300 and/or electrode lead 200 through distal end 412 of dilator 400.

FIG. 4C is a close-up, partial cross-sectional view of alignment marker 406 at proximal region 402 of dilator 400, as indicated by circle C. The textured surface of proximal region 402 of dilator 400 is omitted for brevity. Alignment marker 406 is used to visually indicate when the cutting edge of trephine 500 is aligned with distal region 404 of dilator 400, e.g., with point 410 of dilator 400 or distal end 412 of dilator 400, such that the surgeon will know when further distal movement of trephine 500 relative to dilator 400 will begin cutting tissue. As shown in FIG. 4C, alignment marker 406 may include groove 414 extending circumferentially about an outer surface of dilator 400. Accordingly, alignment marker 406 may visually indicate alignment between the cutting edge of trephine 500 and distal region 404 of dilator 400 when the proximal end of trephine 500 is aligned with groove 414. Alternatively, rather than a groove, a smooth section between textured sections may be used.

Referring now to FIGS. 4D and 4E, an alternative exemplary dilator is provided. Dilator 450 may be constructed similar to dilator 400 with similar components having like-prime reference numerals. For example, proximal region 402′, fillet 403′, distal region 404′, textured surfaces 405′, point 410′, and distal end 412′ correspond with proximal region 402, fillet 403, distal region 404, textured surfaces 405, point 410, and distal end 412. Dilator 450 differs from dilator 400 in that dilator 450 includes alignment marker 452, as described in further detail below with regard to FIG. 4E. Additionally, dilator 450 also may include smooth section 451, e.g., a non-textured surface, extending proximally from textured surface 405′ to fillet 403′, as shown in FIG. 4D.

FIG. 4E is a close-up, partial cross-sectional view of alignment marker 452 at proximal region 402′ of dilator 450, as indicated by circle E. The textured surface of proximal region 402′ of dilator 450 is omitted for brevity. Alignment marker 452 is used to visually indicate when the cutting edge of trephine 500 is aligned with distal region 404′ of dilator 450, e.g., with point 410′ of dilator 450 or distal end 412′ of dilator 450, such that the surgeon will know when further distal movement of trephine 500 relative to dilator 450 will begin cutting tissue. As shown in FIG. 4E, alignment marker 452 may include smooth section 454 extending between textured surfaces 405′ of dilator 450. Accordingly, alignment marker 452 may visually indicate alignment between the cutting edge of trephine 500 and distal region 404′ of dilator 450 when the proximal end of trephine 500 is aligned with smooth section 454.

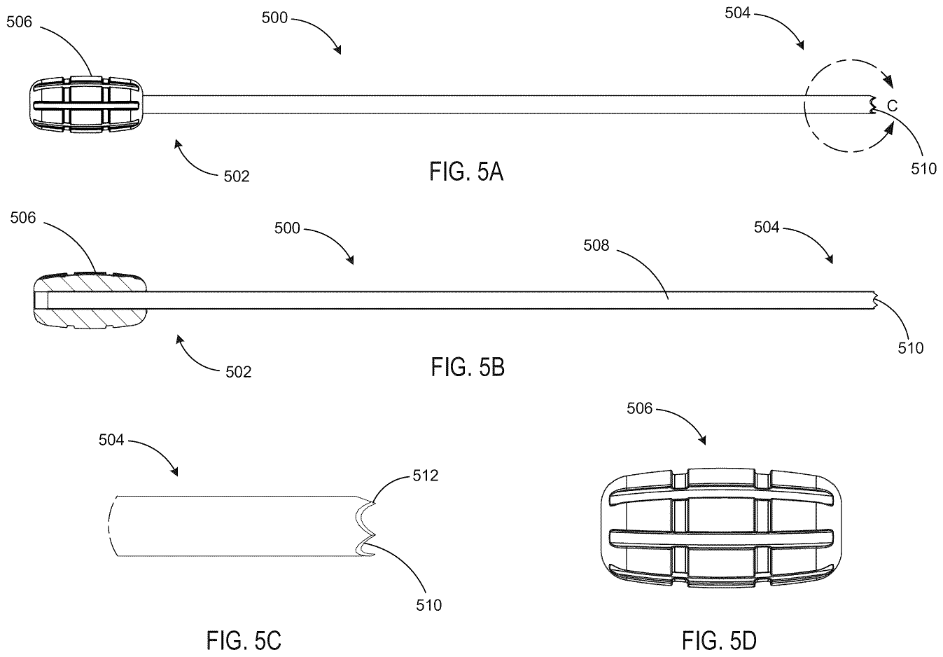

Referring now to FIGS. 5A to 5D, an exemplary trephine is provided. As shown in FIG. 5A, trephine 500 includes proximal region 502 which may include handle 506, and distal region 504 having cutting edge 510. FIG. 5B is a cross-sectional view of trephine 500. Trephine 500 may be made of a rigid material, e.g., stainless steel, and have lumen 508 extending through an entire length of trephine 5000. Lumen 508 is sized and shaped to receive the proximal end of dilator 400, such that trephine 500 is rotatably slidable over dilator 400. For example, the proximal end of dilator 400 may be fed through lumen 508 of trephine 500, such that trephine 500 may be advanced distally relative to dilator 400, sheath 300, and electrode lead 200. In addition, lumen 508 is further sized and shaped to receive distal fixation elements 214 in a collapsed state therethrough.

FIG. 5C is a close-up, partial cross-sectional view of distal region 504 of trephine 500, as indicated by circle C. As shown in FIG. 5C, cutting edge 510 of trephine 500 may include a plurality of proximally extending concave edges circumferentially disposed along a distal end of trephine 500. For example, cutting edge 510 of trephine 500 may include a plurality of C-shapes circumferentially arranged around the distal end such that the corresponding ends of each C-shape meets with an adjacent C-shape, e.g., forming a point at the apex. As such, the conjoining of adjacent C-shapes forms a pointed edge shape. Moreover, cutting edge 510 may include an angled surface 512, e.g., a blade, extending from the outer surface of trephine 500 towards the inner surface of trephine 512 at the distal end of trephine 500 to facilitate cutting of surrounding tissue by axially and rotational movement of trephine 500. For example, the cross-sectional area of trephine 500 may decrease toward the distal end of trephine 500 at cutting edge 510. Cutting edge 510 is sufficiently sharp to cut tissue, e.g., tissue surrounding distal fixation elements 214 of electrode lead 200, to thereby dislodge electrode lead 200 from the surrounding tissue. Cutting edge 510 further may be sharp enough to cut at least a portion of distal fixation elements 214 as trephine is rotatably advanced distally over distal fixation elements 214, such that the remainder of distal fixation elements 214 are disposed within lumen 508 of trephine 500. Alternatively, trephine 500 may include a plurality of proximally extending V-shaped edges circumferentially disposed along a distal end of trephine 500. As will be understood by a person having ordinary skill in the art, cutting edge 510 may be formed of other shapes for safely and effectively cutting tissue and at least a portion of distal fixation elements 214.

FIG. 5D illustrates handle 506 at proximal region 502 of trephine 500. Handle 506 may include a textured surface to facilitate gripping by the surgeon during the lead extraction procedure, e.g., to advance trephine 500 distally relative to dilator 400 and electrode lead 200 while simultaneously rotating trephine 500. As described above, alignment of trephine 500 with the distal end of dilator 400 may be visually indicated by aligning the proximal end of trephine 500, e.g., the proximal end of handle 506, with alignment mark 406.

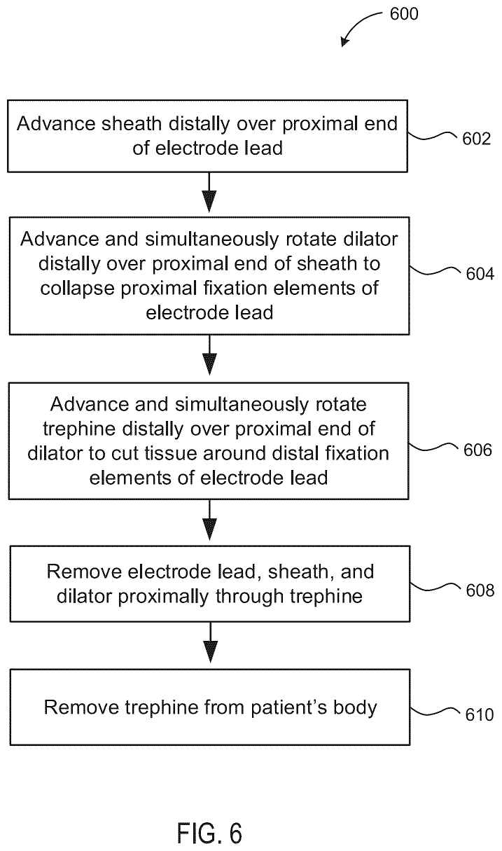

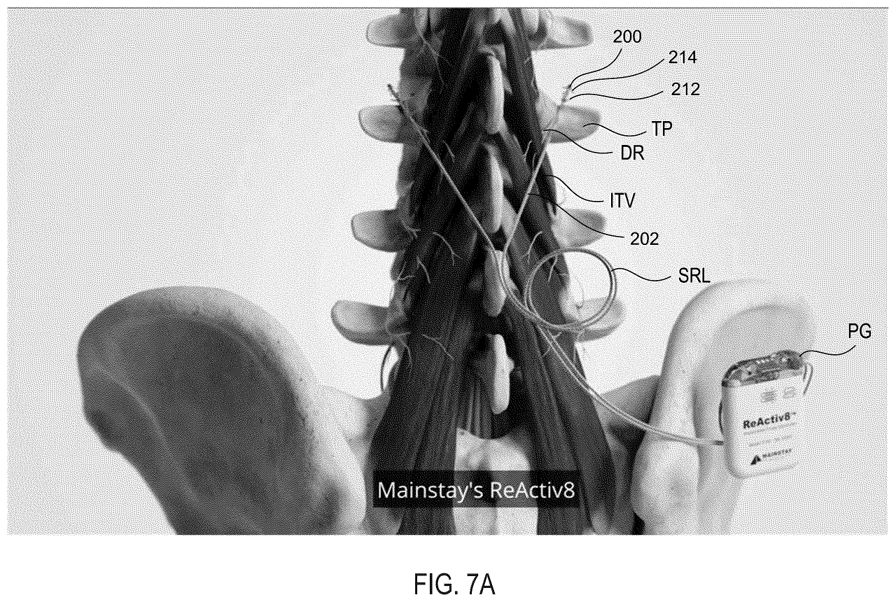

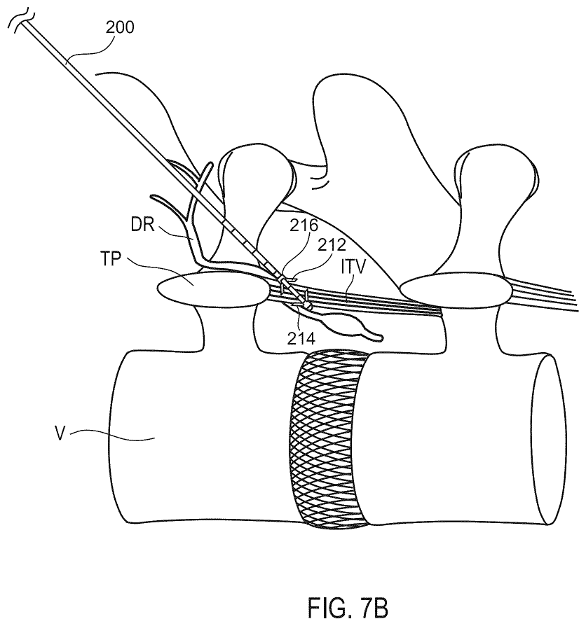

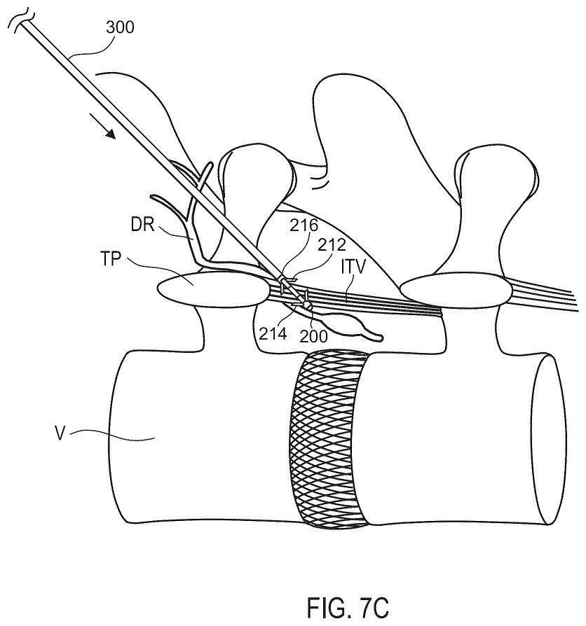

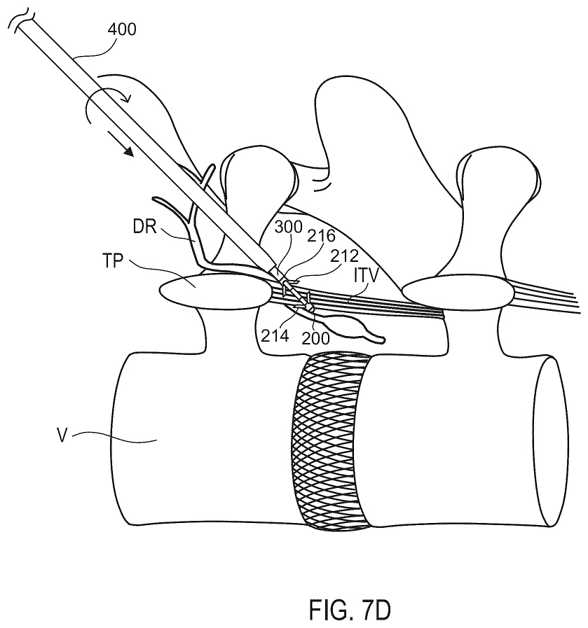

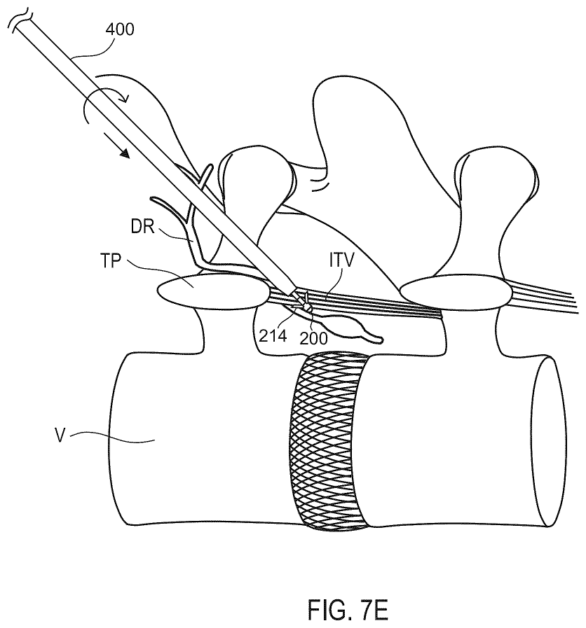

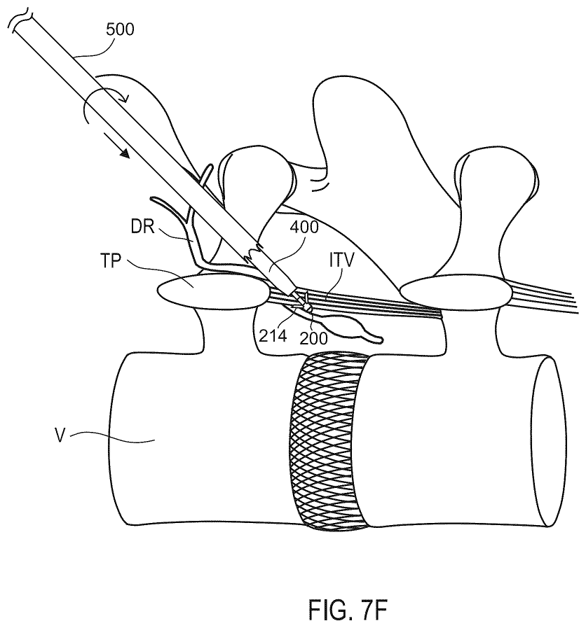

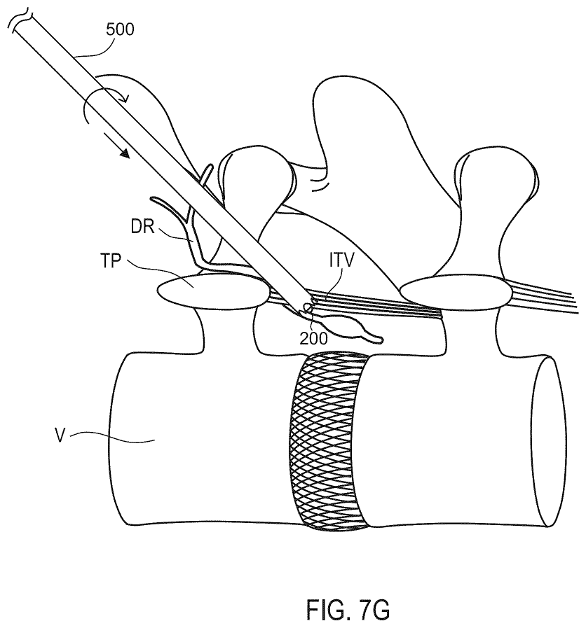

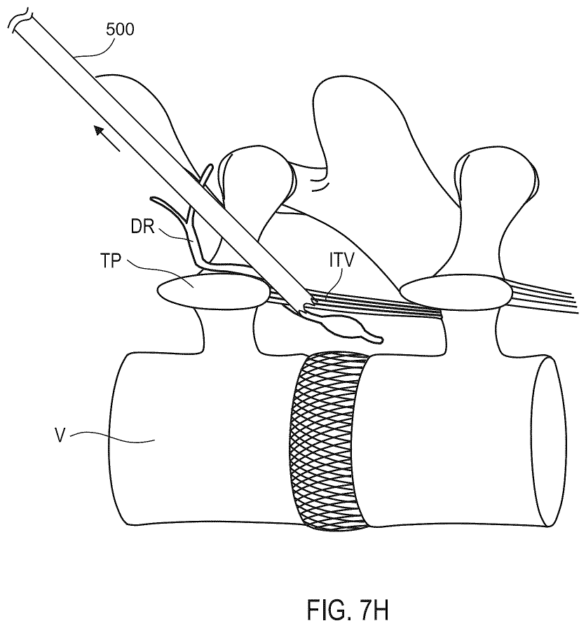

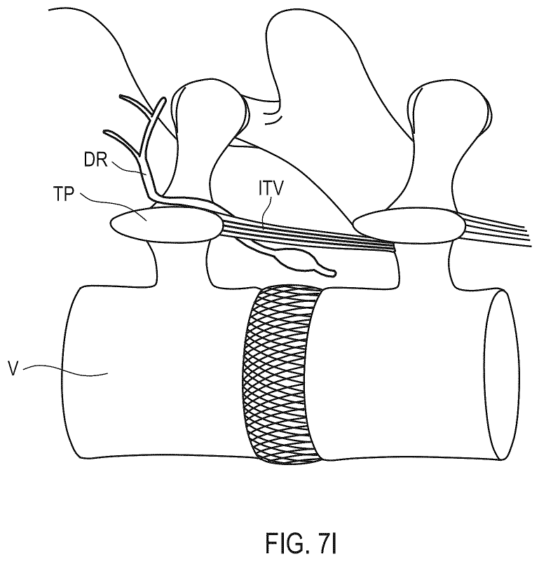

Referring now to FIG. 6, an exemplary method for extracting implanted electrode lead 200 using system 100 is provided. Some of the steps of method 700 may be further elaborated by referring to FIGS. 7A to 7I. Initially, as shown in FIG. 7A, one or more electrode leads 200 may be coupled to pulse generator PG for providing electrostimulation therapy to tissue associated with one or more spine stabilizing muscles associated with local segmental control of a lumbar spine within a back of a patient. Illustratively, FIG. 7A shows the ReActiv8® system available from Mainstay Medical Limited of Dublin, Ireland. As shown in FIG. 7A, pulse generator PG may be implanted in the patient, e.g., an implantable pulse generator (IPG), within the lower back of the patient such that electrode lead 200 is fully implanted. Alternatively, pulse generator PG may be worn externally by the patient, such that electrode lead 200 is partially implanted and extends from pulse generator PG percutaneously toward the target stimulation site. Either way, the target stimulation site may be above traverse process TP adjacent to dorsal root DR within the plane of intertransversarii ITV, along the patient's spinal column in the lumbar region.

Pulse generator PG is configured to generate pulses such that electrodes 204, 206, 208, 210 deliver neuromuscular electrical stimulation (“NMES”) to target tissue. In one embodiment, the electrodes are positioned to stimulate a peripheral nerve where the nerve enters skeletal muscle, which may be one or more of the multifidus, transverse abdominus, quadratus lumborum, psoas major, internus abdominus, obliquus externus abdominus, and erector spinae muscles. Such stimulation may induce contraction of the muscle to restore neural control and rehabilitate the muscle, thereby improving muscle function of local segmental muscles of the lumbar spine, improving lumbar spine stability, and reducing back pain.

Exemplary stimulation parameters in accordance with aspects of the present disclosure are now described. Preferably, such stimulation parameters are selected and programmed to induce contraction of muscle to restore neural control and rehabilitate muscle associated with control of the spine, thereby improving lumbar spine stability and reducing back pain. As used in this specification, “to restore muscle function” means to restore an observable degree of muscle function as recognized by existing measures of patient assessment, such as the Oswestry Disability Index (“ODI”) as described in Lauridsen et al., Responsiveness and minimal clinically important difference for pain and disability instruments in low back pain patients, BMC Musculoskeletal Disorders, 7:82-97 (2006), the European Quality of Life Assessment 5D (“EQ-5D”) as described in Brazier et al., A comparison of the EQ-5D and SF-6D across seven patient groups, Health Econ. 13:873-884 (2004), or a Visual Analogue Scale (“VAS”) as described in Hagg et al., The clinical importance of changes in outcome scores after treatment for chronic low back pain, Eur Spine J 12:12-20 (2003). In accordance with one aspect of the present disclosure, “to restore muscle function” means to observe at least a 15% improvement in one of the foregoing assessment scores within 30-60 days of initiation of treatment. The stimulation parameters may be programmed into pulse generator PG, and/or may be adjusted in the pulse generator 300 responsive to (i) stimulation commands transferred from the activator or (ii) programming data transferred from the external programmer.

The stimulation parameters include, for example, pulse amplitude (voltage or current), pulse width, stimulation rate, stimulation frequency, ramp timing, cycle timing, session timing, and electrode configuration, including commands to start or stop a treatment session. In one embodiment, pulse amplitude is programmed to be adjustable between 0 and 7 mA. In a preferred embodiment, pulse amplitude is programmed to be between about 2-5 mA, 2.5-4.5 mA, or 3-4 mA, and preferably about 3.5 mA. In one embodiment, pulse width is programmed to be adjustable between 25 and 500 μs. In a preferred embodiment, pulse width is programmed to be between about 100-400 μs, 150-350 μs, or 200-300 μs, and preferably about 350 μs. In one embodiment, stimulation rate is programmed to be adjustable between 1 and 40 Hz. In a preferred embodiment, stimulation rate is programmed to be between about 5-35 Hz, 10-30 Hz, or 15-20 Hz, and preferably about 20 Hz. In one embodiment, on ramp timing is programmed to be adjustable between 0 and 5 s. In a preferred embodiment, on ramp timing is programmed to be between about 0.5-4.5 s, 1-4 s, 1.5-3.5 s, or 2-3 s, and preferably about 2.5 s. In one embodiment, off ramp timing is programmed to be adjustable between 0 and 5 s. In a preferred embodiment, off ramp timing is programmed to be between about 0.5-4.5 s, 1-4 s, 1.5-3.5 s, or 2-3 s, and preferably about 2.5 s. In one embodiment, cycle-on timing is programmed to be adjustable between 2 and 20 s. In a preferred embodiment, cycle-on timing is programmed to be between about 4-18 s, 6 -16 s, 8-14 s, 9-13 s, or 10-12 s and preferably about 10 s. In one embodiment, cycle-off timing is programmed to be adjustable between 20 and 120 s. In a preferred embodiment, cycle-off timing is programmed to be between about 30-110 s, 40-100 s, 50-90 s, 55-85 s, 60-80 s, or 65-75 s and preferably about 70 s. In one embodiment, session timing is programmed to be adjustable between 1 and 60 min. In a preferred embodiment, session timing is programmed to be between about 5 -55 min, 10-50 min, 15-45 min, 20-40 min, or 25-35 min, and preferably about 30 min.

As shown in FIG. 7A, electrode lead 200 may include strain relief loop SRL for reducing axial forces on proximal and distal fixation elements 212, 214 of electrode lead 200, as described in U.S. Pat. No. 10,195,419 to Shiroff, assigned to the assignee of the present disclosure, and incorporated herein in its entirety by reference. In order to begin the extraction procedure of electrode lead 200 from the patient, an incision is first made adjacent to the connection of electrode lead 200 and pulse generator PG, and the proximal end of electrode lead 200 is decoupled from pulse generator PG such that the proximal end of electrode lead 200 is exposed from the patient, as shown in FIG. 7B. When pulse generator PG is worn externally by the patient, the proximal end of electrode lead 200 may be decoupled from pulse generator PG external to the patient, and an incision may be made adjacent to the entry point of electrode lead 200 into the patient to permit percutaneous passage of lead extraction system 100.

At step 602, the proximal end of electrode lead 200 may be fed through lumen 306 of sheath 300 such that sheath 300 may be advanced distally relative to electrode lead 200 until the distal end of sheath 300 abuts collar 216 of electrode lead 200, as shown in FIG. 7C. At step 604, the proximal end of sheath 300 and electrode lead 200 may be fed through lumen 408 of dilator 400, such that dilator 400 may be advanced distally while simultaneously rotated along sheath 300, as shown in FIG. 7D. As shown in FIG. 7E, dilator 400 may be rotatably and axially advanced distally relative to electrode lead 200 until distal end 412 contacts and causes proximal fixation elements 212 of electrode lead 200 to transition to its collapsed configuration toward lead body 202 of electrode lead 200 within lumen 408 of dilator 400. Successful collapse of proximal fixation elements 212 within dilator 400 may be observed via, e.g., fluoroscopy. As described above, in some embodiments, sheath 300 does not need to be advanced over electrode lead 200, and thus, the proximal end of electrode lead 200 may be fed directly into lumen 408 of dilator 400, such that dilator 400 may be rotatably and axially advanced directly over electrode lead 200 until distal end 412 of dilator 400 collapses proximal fixation elements 212 of electrode lead 200, as shown in FIG. 7E.

At step 606, the proximal end of dilator 400, sheath 300, and electrode lead 200 may be fed through lumen 508 of trephine 500, such that trephine 500 may be advanced distally while simultaneously rotated along dilator 400, as shown in FIG. 7F. By observing when the proximal end of trephine 500, e.g., the proximal end of handle 506, is aligned with alignment marker 406 of dilator 400, the surgeon may know when cutting edge 510 is aligned with the distal end of dilator 400 such that further axially movement of trephine 500 relative to dilator 400 will begin cutting tissue surrounding the distal region of electrode lead 200.

As shown in FIG. 7G, trephine 500 may be rotatably and axially advanced distally relative to dilator 400 and electrode lead 200 beyond distal end 412 of dilator 400 such that cutting edge 510 of trephine 500 cuts tissue surrounding distal fixation elements 214. As described above, cutting edge 510 may also cut at least a portion of distal fixation elements 214 as cutting edge 510 is rotatably and axially advanced over distal fixation elements 214. Trephine 500 may be rotatably and axially advanced relative to electrode lead 200 until distal fixation elements 214, or what remains of distal fixation elements 214, is disposed within lumen 508 of trephine 500. Successful collapse of distal fixation elements 214 within trephine 500 may be observed via, e.g., fluoroscopy.

At step 608, electrode lead 200, sheath 300 (if used), and dilator 400 may be removed from the patient by being retracted proximally through lumen 508 of trephine 500, as shown in FIG. 7H. At step 610, trephine 500 may be removed from the patient's body as shown in FIG. 7I.

While various illustrative embodiments of the disclosure are described above, it will be apparent to one skilled in the art that various changes and modifications may be made therein without departing from the disclosure. For example, system 100 may be used to extract various types of neuromodulation leads, e.g., leads implanted for peripheral neuromodulation or leads used for spinal cord stimulation, as well as leads implanted in other parts of the body other than the lower back, e.g., in peripheral musculoskeletal, fascial, or visceral areas of the body. Further, system 100 may be used to extract neuromodulation leads having various fixation element configurations, e.g., single or multiple fixation elements such as tines, hooks, barbs, coils, etc., as well as fixation elements extending from the lead body at various angles, e.g., proximally extending, distally extending, perpendicular, or any combination thereof. The appended claims are intended to cover all such changes and modifications that fall within the true scope of the disclosure.

What is claimed:

1. A method for extracting an electrode lead implanted at an implantation site in a patient's body via at least one fixation element, the method comprising:
exposing a proximal end of the electrode lead from the patient's body;
advancing a dilator distally over the proximal end of the electrode lead to thereby separate tissue surrounding the electrode lead;
advancing a trephine distally over a proximal end of the dilator to position a distal end of the trephine adjacent to the at least one fixation element;
operating the trephine such that a cutting edge at the distal end of the trephine separates tissue surrounding the at least one fixation element to thereby dislodge the electrode lead from the implantation site; and
removing the electrode lead, the dilator, and the trephine from the patient's body.

2. The method of claim 1, wherein exposing the proximal end of the electrode lead from the patient's body comprises decoupling the proximal end of the electrode lead from a pulse generator.

3. The method of claim 1, wherein advancing the dilator distally over the proximal end of the electrode lead comprises rotating the dilator as the dilator is advanced distally to facilitate separation of the tissue surrounding the electrode lead.

4. The method of claim 1, wherein advancing the dilator distally over the proximal end of the electrode lead comprises gripping a textured surface disposed at a proximal region of the dilator.

5. The method of claim 1, wherein advancing the trephine distally to position the distal end of the trephine adjacent to the at least one fixation element comprises aligning the cutting edge of the trephine and a distal end of the dilator.

6. The method of claim 5, further comprising verifying the alignment of the cutting edge of the trephine and the distal end of the dilator.

7. The method of claim 6, wherein verifying the alignment of the cutting edge of the trephine and the distal end of the dilator comprises observing an alignment of a proximal end of the trephine with an alignment marker disposed at a proximal region of the dilator.

8. The method of claim 1, wherein operating the trephine comprises rotating the trephine as the trephine is advanced distally to facilitate separation of the tissue surrounding the at least one fixation element.

9. The method of claim 1, wherein operating the trephine such that the cutting edge separates tissue surrounding the at least one fixation element comprises operating the trephine such that the cutting edge cuts the tissue surrounding the at least one fixation element to thereby dislodge the electrode lead from the implantation site.

10. The method of claim 1, wherein operating the trephine such that the cutting edge separates tissue surrounding the at least one fixation element comprises operating the trephine such that the cutting edge cuts at least a portion of the at least one fixation element to facilitate dislodgement of the electrode lead.

11. The method of claim 1, wherein operating the trephine comprises advancing the trephine until the at least one fixation element is disposed within a lumen of the trephine.

12. The method of claim 11, further comprising verifying that the at least one fixation element is disposed within the lumen of the trephine via fluoroscopy.

13. The method of claim 1, wherein the at least one fixation element comprises a proximal fixation element and a distal fixation element, and
wherein advancing the dilator distally comprises advancing the dilator distally over the proximal fixation element to thereby collapse the proximal fixation element within a lumen of the dilator.

14. The method of claim 13, further comprising verifying collapse of the proximal fixation element within the lumen of the dilator via fluoroscopy.

15. The method of claim 13, wherein operating the trephine comprises operating the trephine such that the cutting edge separates tissue surrounding the distal fixation element to thereby dislodge the electrode lead from the implantation site.

16. The method of claim 13, further comprising:
prior to advancing the dilator distally over the proximal end of the electrode lead, advancing a sheath distally over a proximal end of the electrode lead until a distal end of the sheath abuts a collar of the proximal fixation element,
wherein advancing the dilator distally over the proximal end of the electrode lead comprises advancing the dilator distally over the proximal end of the sheath.

17. The method of claim 1, wherein removing the electrode lead, the dilator, and the trephine from the patient's body comprises:
removing the electrode lead and dilator proximally through the lumen of the trephine; and
removing the trephine from the patient's body.

18. The method of claim 1, wherein the implantation site comprises at least one of a multifidus, transverse abdominus, quadratus lumborum, psoas major, internus abdominus, obliquus externus abdominus, or erector spinae muscle.

19. The method of claim 1, further comprising:
prior to advancing the dilator distally over the proximal end of the electrode lead, advancing a sheath distally over the proximal end of the electrode lead until a distal end of the sheath abuts the at least one fixation element,
wherein advancing the dilator distally over the proximal end of the electrode lead comprises advancing the dilator distally over the proximal end of the sheath.

20. The method of claim 1, further comprising inserting a stylet within a lumen of the electrode lead to facilitate extraction of the electrode lead.
